(19) United States
(12) Patent Application Publication
(10) Pub. No.: US 2026/0283967 A1
(43) Pub. Date: Sep. 24, 2026

(54) FAT-SOLUBLE NUTRIENT MICROCAPSULE AS WELL AS PREPARATION METHOD AND USE THEREOF

(71) Applicants: XINCHANG NHU VITAMINS COMPANY LTD., Shaoxing (CN); ZHEJIANG NHU CO., LTD., Shaoxing (CN)

(72) Inventors: Xiang Liu, Shaoxing (CN); Shihua Hao, Shaoxing (CN); Jiandong Li, Shaoxing (CN); Qilei Zhang, Hangzhou (CN); Jincheng Cheng, Shaoxing (CN); Xingyu Zhang, Shaoxing (CN); Qiyu Guo, Shaoxing (CN); Qiaoqiao Liu, Shaoxing (CN)

(21) Appl. No.: 19/573,353

(22) Filed: Mar. 20, 2026

(30) Foreign Application Priority Data
Mar. 21, 2025 (CN) 202510345943.6

(51) Int. Cl. A61K 9/50; A23P 10/35

(52) U.S. Cl. CPC A61K 9/5057; A23P 10/35; A61K 9/5015; A61K 9/5036

(57) ABSTRACT

The present disclosure relates to a fat-soluble nutrient microcapsule as well as a preparation method and use thereof. The present disclosure provides a fat-soluble nutrient microcapsule. The fat-soluble nutrient microcapsule comprises the following components: a fat-soluble nutrient, a hydrophilic colloid, a co-emulsifier and starch, wherein the hydrophilic colloid comprises gelatin with a gel strength of 125 to 270 Bloom g; the co-emulsifier comprises glycerin and/or sorbitol. The fat-soluble nutrient microcapsule provided by the present disclosure has good pressure resistance and stability, can meet tableting requirements, and can be stored for a long time. Meanwhile, the present disclosure further provides a preparation method of the fat-soluble nutrient microcapsule, which allows for production of the fat-soluble nutrient microcapsule with a dense and void-free internal structure and having good in pressure resistance and stability.

### CROSS-REFERENCE TO RELATED APPLICATIONS

The present application claims priority to the Chinese Patent Application No. 202510345943.6 filled on Mar. 21, 2025, which is incorporated herein by reference in its entirety.

### TECHNICAL FIELD

The present disclosure belongs to the field of food and health-care product additives and specifically relates to a fat-soluble nutrient microcapsule, a preparation method therefor, and use thereof, and more specifically to a pressure-resistant fat-soluble nutrient microcapsule, a preparation method therefor, and use thereof.

### BACKGROUND

The fat-soluble nutrient according to the present disclosure includes primarily fat-soluble vitamins, carotenoids, and coenzyme Q10 (CoQ10). Vitamins are a group of trace organic regulatory substances that must be obtained by humans and animals from diets to maintain normal physiological functions, and they play a vital role in growth, metabolism, and development of the body. Carotenoids are a collective term for a significant class of natural pigments, which could enhance animal fecundity and immune functions and impart various physiological functions including the antioxidation, pigmentation, and enhanced intercellular communication via gap junctions. Coenzyme Q10 is a fat-soluble quinone compound capable of activating human cells and crucial for cellular energy production. It functions to boost immunity, enhance the antioxidant capacity, delay aging, increase vitality, etc. It is widely used in medicine as an adjunctive therapy for cardiovascular diseases and is extensively utilized worldwide as a nutritional dietary supplement and a food additive.

Fat-soluble vitamins, carotenoids, and coenzyme Q10 are all very unstable substances—extremely sensitive to light, heat, and oxygen—and are unsuitable for direct addition to food and health-care products. Consequently, researchers and relevant enterprises have developed a variety of methods to stabilize these fat-soluble nutrients. A common practice is to prepare such fat-soluble nutrients into microcapsules for subsequent use as additives.

The fat-soluble nutrient microcapsule is typically prepared by dissolving the nutrient and other fat-soluble core materials into oils and fats or an organic solvent to form an oil phase; then mixing the oil phase with an aqueous phase containing a water-soluble wall material; emulsifying the mixture by such a method as high-pressure homogenization, high-speed shear, high-speed jetting, ultrasonic cavitation or grinding, followed by spray granulation and drying to obtain microcapsules. The fat-soluble nutrient is encapsulated in a tiny capsule formed of a continuous wall material and is shielded from direct contact with the environment, thereby maintaining its stability during storage, transport, and use. However, studies have shown that when microcapsules are directly compressed into tablets, they are prone to rupture their insufficient mechanical strength. Furthermore, stability studies have shown a decrease in nutrient content, suggesting that the rupture of microcapsules results in oxidative degradation of the fat-soluble nutrient.

To enhance the stability of fat-soluble nutrient microcapsules, technicians typically make improvements to the microcapsules, including selection of the wall material, the oil phase or other excipients, incorporation of supportive structures, and refinement of the preparation process to improve the pressure resistance of the microcapsules, thereby increasing their stability in applications. For example, CN101873848B, which modifies the wall material of the microcapsule, describes a formulation of a lipophilic health ingredient, comprising the lipophilic health ingredient and a protective colloid, wherein the protective colloid is a modified starch with the emulsifying capacity, and the lipophilic health ingredient is selected from the group consisting of vitamin A, CoQ10, and esters thereof. CN103549157B describes a method for preparing hydrophobic vitamin microcapsules, comprising the steps of: adding a proteolytic enzyme to a vitamin-containing emulsion formed by emulsifying and homogenizing a wall material and a core material; then subjecting the mixture to granulation and a crosslinking reaction; and finally drying the resultant to obtain the hydrophobic vitamin microcapsule. U.S. Pat. No. 8,685,446B2 describes a multi-wall microcapsule encapsulated with multiple protective colloids.

A common prior-art practice of improving the pressure resistance of the fat-soluble nutrient microcapsules during the emulsification in the preparation process for microcapsules is to compound multiple wall materials or specific encapsulating materials with a dispersing agent. For example, CN106074462B describes that gelatin and gum Arabic are compounded to encapsulate lycopene and then homogenized using a high-pressure homogenizer, and the homogenized emulsion is charged into a temporary storage tank, vacuumized, stirred, and subjected to one-step granulation by the spray-starch fluidized bed drying process to obtain lycopene microcapsules. By selecting different encapsulating wall materials, combining them with a dispersing agent and taking advantage of their synergistic effect, CN112189846A employs specific encapsulating materials and a dispersing agent to overcomes the inherent defects in applications of oils and fats and fat-soluble functional ingredient raw materials by the microencapsulation technology. It resolves the problems about oil leakage, seepage, instability and the like during the tableting of the fat-soluble functional ingredients. Consequently, the high-oil-loaded microparticles exhibit enhanced mechanical strength and stability and are pressure-resistant without oil leakage, and thus have good application prospects in preparation of tablet or capsule products. However, the emulsification method, such as high-speed shear and high-pressure homogenization, has the following drawbacks: 1) the emulsification procedure is typically conducted in batches in an open environment and each batch is time-consuming, which makes it prone to introducing a large number of air bubbles, leading to deterioration of the fat-soluble nutrients; and 2) the internal structure of the resulting microparticle samples is not dense enough and contains a large number of voids, which compromises the pressure resistance of the product. Owing to these factors, conventional fat-soluble nutrient microcapsule products suffer from partial loss during preparation and are susceptible to rupture during tableting, which exposes the fat-soluble nutrients directly to the internal tablet environment including the excipients, thereby impairing the stability of the fat-soluble nutrient during the tablet storage.

### SUMMARY

### Problem to be Solved by the Invention

In view of the problems about poor pressure resistance and instability of fat-soluble nutrient microcapsules in practical applications in the prior art, the present disclosure aims to provide a pressure-resistant fat-soluble nutrient microcapsule, a preparation method therefor, and use thereof.

### Solution to Problem

To address the above technical problem, the present disclosure provides the following technical solutions:

[1]. A fat-soluble nutrient microcapsule, wherein the fat-soluble nutrient microcapsule comprises the following components: a fat-soluble nutrient, a hydrophilic colloid, a co-emulsifier, and starch; wherein
the hydrophilic colloid comprises gelatin having a gel strength of 125 to 270 Bloom g; and
the co-emulsifier comprises glycerin and/or sorbitol.

[2]. The fat-soluble nutrient microcapsule according to [1], wherein the fat-soluble nutrient microcapsule comprises 30 to 60 mass % of the hydrophilic colloid and 1 to 5 mass % of the co-emulsifier, based on a total dry weight of the fat-soluble nutrient microcapsule.

[3]. The fat-soluble nutrient microcapsule according to [1] or [2], wherein the fat-soluble nutrient comprises any one or more of vitamin A, an ester of vitamin A, vitamin E, an ester of vitamin E, vitamin D2, vitamin D3, vitamin K, coenzyme Q10, curcumin, β-carotene, lutein, canthaxanthin, an ester of lutein, lycopene, astaxanthin, and a polyunsaturated fatty acid.

[4]. The fat-soluble nutrient microcapsule according to any one of [1] to [3], wherein the fat-soluble nutrient microcapsule comprises 10 to 30 mass % of the fat-soluble nutrient based on a total dry weight of the fat-soluble nutrient microcapsule.

[5]. The fat-soluble nutrient microcapsule according to any one of [1] to [4], wherein the starch comprises any one or more of potato starch, corn starch, waxy corn starch, wheat starch, barley starch, rye starch, rice starch, sorghum starch, white sweet potato starch, tapioca starch, orange sweet potato starch, and legume starch.

[6]. The fat-soluble nutrient microcapsule according to any one of [1] to [5], wherein the fat-soluble nutrient microcapsule comprises 15 to 25 mass % of the starch based on a total dry weight of the fat-soluble nutrient microcapsule.

[7]. The fat-soluble nutrient microcapsule according to any one of [1] to [6], wherein the fat-soluble nutrient microcapsule further comprises a filler; the filler comprises any one or more of maltodextrin, glucose, white granulated sugar, crystalline fructose, maltose, inositol, and xylitol; and/or the fat-soluble nutrient microcapsule comprises 5 to 20 mass % of the filler based on a total dry weight of the fat-soluble nutrient microcapsule.

[8]. The fat-soluble nutrient microcapsule according to any one of [1] to [7], wherein the fat-soluble nutrient microcapsule further comprises an antioxidant; the antioxidant comprises any one or more of tocopherol, butylated hydroxytoluene, butylated hydroxyanisole, tea polyphenol, propyl gallate, a rosemary extract, a licorice antioxidant, phytic acid, ascorbic acid, sodium ascorbate, D-isoascorbic acid, sodium D-isoascorbate, ascorbyl palmitate, and lecithin; and/or the fat-soluble nutrient microcapsule comprises 0.1 to 10 mass % of the antioxidant based on a total dry weight of the fat-soluble nutrient microcapsule.

[9]. The fat-soluble nutrient microcapsule according to any one of [1] to [8], wherein the fat-soluble nutrient microcapsule further comprises less than or equal to 5 mass % of water.

[10]. A preparation method for the fat-soluble nutrient microcapsule according to any one of [1] to [9], wherein the preparation method comprises the following steps:
S1: mixing a fat-soluble nutrient, a hydrophilic colloid, a co-emulsifier, an optional filler, and an optional antioxidant with water, and stirring while vacuumizing to afford a crude emulsion;
S2: shearing the crude emulsion while continuously vacuumizing to afford an emulsified solution or dispersion; and
S3: subjecting the emulsified solution or dispersion to one-step granulation by a spray-starch fluidized bed drying process to afford the fat-soluble nutrient microcapsule.

[11]. The preparation method according to [10], wherein in the step S1, the stirring is conducted at a speed of 1.0 to 1.5 m/s; and/or the stirring is conducted at a temperature of 60° C. to 70° C.; and/or the stirring is conducted for 30 to 60 min.

[12]. The preparation method according to [10] or [11], wherein in the step S2, the shearing is conducted at a speed of greater than or equal to 19.6 m/s; and/or the shearing is conducted at a temperature of 60° C. to 65° C.; and/or the shearing is conducted for 20 to 40 min.

[13]. The preparation method according to any one of [10] to [12], wherein in the step S3, when the emulsified solution or dispersion is subjected to the one-step granulation by the spray-starch fluidized bed drying process, fluidized drying is conducted in stages, wherein a first stage is conducted at a temperature of 18° C. to 40° C. for 10 to 20 min, and a second stage is conducted at a temperature of 40° C. to 60° C. for 40 to 60 min.

[14]. Use of the fat-soluble nutrient microcapsule according to any one of [1] to [9] and/or a fat-soluble nutrient microcapsule obtained by the preparation method according to any one of [10] to [13] in preparation of a food or health-care product.

### Advantageous Effects of the Invention

By implementing the above technical solutions, the present disclosure achieves the following advantageous effects:

First of all, the present disclosure provides a fat-soluble nutrient microcapsule having good pressure resistance and stability. The experimental data show that the fat-soluble nutrient microcapsules provided in the present disclosure withstand 100% deformation under 25N pressure while remaining intact (without rupture); after being left for 12 months under 25° C., RH75% conditions, the content retention rate of the fat-soluble nutrient is greater than 95%; and after being left for 12 months under 25° C., RH75% conditions following direct compression into tablets, the content retention rate of the fat-soluble nutrient is still greater than 95%, which is sufficient to meet the tableting requirements.

Furthermore, the present disclosure also provides a preparation method for the fat-soluble nutrient microcapsules. This preparation method is simple in process and suitable for mass production. Moreover, the preparation method provided in the present disclosure allows for production of fat-soluble nutrient microcapsules with a dense and void-free internal structure and having good pressure resistance and stability.

Owing to their good pressure resistance and stability, the fat-soluble nutrient microcapsules provided in the present disclosure can also be used as a raw material in processed food or health-care products, and particularly in the preparation of a tablet-type health-care food.

### BRIEF DESCRIPTION OF THE DRAWINGS

FIG. 1: Test results of the pressure resistance of the vitamin A microcapsule in Example 1.

FIG. 2: Test results of the pressure resistance of the vitamin A microcapsule in Example 2.

FIG. 3: Test results of the pressure resistance of the vitamin A microcapsule in Comparative Example 1.

FIG. 4: Test results of the pressure resistance of the vitamin A microcapsule in Comparative Example 2.

FIG. 5: Test results of the pressure resistance of the vitamin A microcapsule in Comparative Example 3.

### DETAILED DESCRIPTION

The embodiments of the present disclosure are described below, but the present disclosure is not limited thereto. The present disclosure is not confined to the elements described below. Various modifications may be made within the scope as claimed in the invention. Moreover, the embodiments or examples obtained by appropriately combining the technical means described respectively in different embodiments or examples are also encompassed in the technical scope of the present disclosure.

In the present disclosure, the term “including”, “having”, “comprising”, or “containing” is intended to be inclusive or open-ended, and does not exclude additional or unrecited elements or methods and steps. At the same time, the term “including”, “having”, “comprising”, or “containing” may also be intended to be close-ended and exclude additional or unrecited elements or methods and steps.

In the present disclosure, the meaning indicated by using “may” involves both the meaning of conducting a certain treatment and the meaning of not conducting a certain treatment.

In the present disclosure, “optional” or “optionally” is used to indicate that some substances, components, execution steps, applied conditions or other factors are used or not used.

In the present disclosure, the numerical range represented by “numerical value A to numerical value B”, “numerical value A-numerical value B” or “numerical range A or more/less” refers to the range including the endpoint values A and B.

In the present disclosure, the term “about” means that all the numerical ranges and parameters used to define the present disclosure are approximate numerical values, and the numerical values involved in the specific examples have already been presented herein as accurately as possible. It should be understood that all the ranges, quantities, numerical values, and percentages used herein are modified by “about”, unless otherwise expressly stated. As used herein, the term “about” generally means that the actual value is within ±5%, ±3%, ±1%, or ±0.5% of a particular numerical value or range. Moreover, the numerical values and numerical ranges used in the present disclosure should all be construed as including systematic errors inevitable in industrial production.

In the present disclosure, references to “some specific/preferred embodiments”, “some other specific/preferred embodiments”, “embodiments” and the like mean that particular elements as described (for example, features, structures, properties and/or characteristics) in relation to this embodiment are included in at least one of the embodiments described here and may or may not exist in other embodiments. Additionally, it should be understood that said elements may be combined in any suitable manner in various embodiments.

In the present disclosure, the units used are all international standard units, and the symbol “%” used refers always to the weight percentage content or mass percentage content, unless otherwise stated.

Unless otherwise defined, other technical and scientific terms used herein have the same meanings as typically understood by one of ordinary skill in the art to which the present disclosure belongs.

### I. First Aspect

In the first aspect, the present disclosure provides a fat-soluble nutrient microcapsule, comprising the following components: a fat-soluble nutrient, a hydrophilic colloid, a co-emulsifier, and starch.

In some embodiments, the fat-soluble nutrient microcapsule further comprises a filler.

In some embodiments, the fat-soluble nutrient microcapsule further comprises an antioxidant.

In some embodiments, the fat-soluble nutrient microcapsule further comprises water.

In some embodiments, the fat-soluble nutrient microcapsule consists of the following components: a fat-soluble nutrient, a hydrophilic colloid, a co-emulsifier, a filler, an antioxidant, and starch.

In some embodiments, the fat-soluble nutrient microcapsule consists of the following components: a fat-soluble nutrient, a hydrophilic colloid, a co-emulsifier, a filler, an antioxidant, starch, and water.

### Fat-Soluble Nutrient

The fat-soluble nutrient according to the present disclosure refers to a substance that is insoluble or substantially insoluble in water under normal temperature (about 25° C.) and normal pressure (about 101.325 kPa) and exerts beneficial effects on life and health.

In some embodiments, the fat-soluble nutrient comprises any one or more of vitamin A, an ester of vitamin A (e.g., retinol acetate, retinol palmitate, retinol linoleate, or retinol propionate), vitamin E, an ester of vitamin E (e.g., tocopheryl acetate, tocopheryl succinate, tocopheryl nicotinate, tocopheryl linoleate, or tocopheryl palmitate), vitamin D2, vitamin D3, vitamin K, coenzyme Q10, curcumin, β-carotene, lutein, canthaxanthin, an ester of lutein (e.g., lutein dipalmitate), lycopene, astaxanthin, and a polyunsaturated fatty acid (e.g., eicosapentaenoic acid (EPA), docosahexaenoic acid (DHA), arachidonic acid (ARA), or γ-linolenic acid (GLA)).

In some embodiments, the fat-soluble nutrient is vitamin A, an ester of vitamin A, vitamin E, an ester of vitamin E, vitamin D2, vitamin D3, vitamin K, coenzyme Q10, curcumin, β-carotene, lutein, canthaxanthin, an ester of lutein, lycopene, astaxanthin or a polyunsaturated fatty acid; and preferably vitamin A.

In some embodiments, based on a total dry weight of the fat-soluble nutrient microcapsule, the fat-soluble nutrient microcapsule contains 10 to 30 mass % of the fat-soluble nutrient; for example, the fat-soluble nutrient microcapsule may contain 10 mass %, 11 mass %, 12 mass %, 13 mass %, 14 mass %, 15 mass %, 16 mass %, 17 mass %, 18 mass %, 19 mass %, 20 mass %, 21 mass %, 22 mass %, 23 mass %, 24 mass %, 25 mass %, 26 mass %, 27 mass %, 28 mass %, 29 mass % or 30 mass % or the like of the fat-soluble nutrient; and preferably the fat-soluble nutrient microcapsule contains 15 to 20 mass % or 18 to 22 mass % of the fat-soluble nutrient.

### Hydrophilic Colloid

The hydrophilic colloid according to the present disclosure refers to a polymer substance capable of interacting with water molecules and forming a colloidal solution.

In some embodiments, the hydrophilic colloid comprises gelatin.

In some embodiments, the hydrophilic colloid is gelatin. The gelatin according to the present disclosure is mainly a proteinaceous substance processed by denaturing and degrading collagen in animal skin or bones. The present disclosure does not particularly limit the preparation method for gelatin. For example, the gelatin may be obtained by the following procedures: washing animal skin or bones to remove impurities and fat thereon; removing the residual fat with hot water or an organic solvent (such as ethanol); rinsing with clean water after acid or alkali treatment; and heating the treated raw material in hot water (e.g., at 60° C. to 100° C.) to hydrolyze collagen into gelatin.

In some embodiments, the gelatin comprises any one or more of porcine gelatin, bovine bone gelatin, and fish gelatin. In some embodiments, the gelatin is porcine gelatin, bovine bone gelatin or fish gelatin; and preferably, the gelatin is porcine gelatin.

In some embodiments, the gelatin has a gel strength of 125 to 275 Bloom g; for example, the gel strength of the gelatin may be 125 Bloom g, 130 Bloom g, 135 Bloom g, 140 Bloom g, 145 Bloom g, 150 Bloom g, 155 Bloom g, 160 Bloom g, 165 Bloom g, 170 Bloom g, 175 Bloom g, 180 Bloom g, 185 Bloom g, 190 Bloom g, 195 Bloom g, 200 Bloom g, 205 Bloom g, 210 Bloom g, 215 Bloom g, 220 Bloom g, 225 Bloom g, 230 Bloom g, 235 Bloom g, 240 Bloom g, 245 Bloom g, 250 Bloom g, 255 Bloom g, 260 Bloom g, 265 Bloom g, 270 Bloom g, 275 Bloom g or the like; and preferably, the gel strength of the gelatin is 200 to 250 Bloom g.

In some embodiments, based on a total dry weight of the fat-soluble nutrient microcapsule, the fat-soluble nutrient microcapsule contains 30 to 60 mass % of the hydrophilic colloid; for example, the fat-soluble nutrient microcapsule may contain 30 mass %, 31 mass %, 32 mass %, 33 mass %, 34 mass %, 35 mass %, 36 mass %, 37 mass %, 38 mass %, 39 mass %, 40 mass %, 41 mass %, 42 mass %, 43 mass %, 44 mass %, 45 mass %, 46 mass %, 47 mass %, 48 mass %, 49 mass %, 50 mass %, 51 mass %, 52 mass %, 53 mass %, 54 mass %, 55 mass %, 56 mass %, 57 mass %, 58 mass %, 59 mass %, 60 mass % or the like of the hydrophilic colloid; preferably, the fat-soluble nutrient microcapsule contains 35 to 50 mass % of the hydrophilic colloid; and more preferably, the fat-soluble nutrient microcapsule contains 38 to 44 mass % of the hydrophilic colloid.

### Co-Emulsifier

The co-emulsifier according to the present disclosure refers to a compound that enables two mutually immiscible substances to be mixed to form an emulsion liquid. It mainly improves the stability of the emulsion by adjusting the fluidity or strength of the interface film and the viscosity or rheology of the emulsion.

In some embodiments, the co-emulsifier comprises glycerin and/or sorbitol; preferably, the co-emulsifier is glycerin and/or sorbitol; more preferably, the co-emulsifier is glycerin or sorbitol; and yet more preferably, the co-emulsifier is glycerin. It has been found in the present disclosure that an appropriate co-emulsifier (e.g., glycerin or sorbitol, especially glycerin) can be combined with gelatin to form a three-dimensional network structure, which increases the toughness and contractile force of microcapsules, endowing the microcapsules with excellent pressure-resistant and rupture-proof properties.

In some embodiments, based on a total dry weight of the fat-soluble nutrient microcapsule, the fat-soluble nutrient microcapsule contains 1 to 5 mass % of the co-emulsifier; for example, the fat-soluble nutrient microcapsule may contain 1 mass %, 2 mass %, 3 mass %, 4 mass %, 5 mass % or the like of the co-emulsifier; preferably, the fat-soluble nutrient microcapsule contains 1 to 5 mass % of the co-emulsifier; and more preferably, the fat-soluble nutrient microcapsule contains 2 to 4 mass % of the co-emulsifier.

### Filler

The filler according to the present disclosure refers to a substance used for filling a material or for increasing the bulk of a material. It can be used to fill voids in microcapsules, and particularly those in the microcapsule walls.

In some embodiments, the filler comprises any one or more of maltodextrin, glucose, white granulated sugar, crystalline fructose, maltose, inositol, and xylitol.

In some embodiments, the filler is maltodextrin, glucose, white granulated sugar, crystalline fructose, maltose, inositol or xylitol; preferably, the filler is white granulated sugar or crystalline fructose; and more preferably, the filler is fructose. The filler selected in the present disclosure can be used to fill the voids in the three-dimensional spatial structure formed by the macromolecular material gelatin and the co-emulsifier to achieve a dense and void-free internal structure, thereby further improving the pressure resistance of the microcapsules.

In some embodiments, based on a total dry weight of the fat-soluble nutrient microcapsule, the fat-soluble nutrient microcapsule contains 5 to 20 mass % of the filler; for example, the fat-soluble nutrient microcapsule may contain 5 mass %, 6 mass %, 7 mass %, 8 mass %, 9 mass %, 10 mass %, 11 mass %, 12 mass %, 13 mass %, 14 mass %, 15 mass %, 16 mass %, 17 mass %, 18 mass %, 19 mass %, 20 mass % or the like of the filler; preferably, the fat-soluble nutrient microcapsule contains 10 to 18 mass % of the filler; and more preferably, the fat-soluble nutrient microcapsule contains 14 to 16 mass % of the filler.

### Antioxidant

The antioxidant according to the present disclosure refers to a substance capable of retarding or inhibiting oxidation reactions.

In some embodiments, the antioxidant comprises any one or more of tocopherol, butylated hydroxytoluene (BHT), butylated hydroxyanisole (BHA), tea polyphenol, propyl gallate, a rosemary extract, a licorice antioxidant, phytic acid, ascorbic acid, sodium ascorbate, D-isoascorbic acid, sodium D-isoascorbate, ascorbyl palmitate, and lecithin.

In some embodiments, the antioxidant comprises an oil phase antioxidant and an aqueous phase antioxidant, wherein the oil phase antioxidant is BHT or tocopherol, and the aqueous phase antioxidant is sodium ascorbate or sodium D-isoascorbate; and preferably, the oil phase antioxidant is BHT, and the aqueous phase antioxidant is sodium ascorbate.

In some embodiments, based on a total dry weight of the fat-soluble nutrient microcapsule, the fat-soluble nutrient microcapsule contains 0.1 to 10 mass % of the antioxidant; for example, the fat-soluble nutrient microcapsule may contain 0.1 mass %, 0.3 mass %, 0.5 mass %, 0.7 mass %, 0.9 mass %, 1 mass %, 1.5 mass %, 2 mass %, 2.5 mass %, 3 mass %, 3.5 mass %, 4 mass %, 4.5 mass %, 5 mass %, 5.5 mass %, 6 mass %, 6.5 mass %, 7 mass %, 7.5 mass %, 8 mass %, 8.5 mass %, 9 mass %, 9.5 mass %, 10 mass % or the like of the antioxidant; and preferably, the fat-soluble nutrient microcapsule contains 0.1 to 1 mass % of the oil phase antioxidant and 1 to 5 mass % of the aqueous phase antioxidant.

### Starch

The starch according to the present disclosure refers to natural polysaccharides that have not been modified by physical, chemical or enzymatic methods, etc.

In some embodiments, the starch comprises any one or more of potato starch, corn starch, waxy corn starch, wheat starch, barley starch, rye starch, rice starch, sorghum starch, white sweet potato starch, tapioca starch, orange sweet potato starch, and legume starch.

In some embodiments, the starch is potato starch, corn starch, waxy corn starch, wheat starch, barley starch, rye starch, rice starch, sorghum starch, white sweet potato starch, tapioca starch, orange sweet potato starch, or legume starch; and preferably corn starch.

In some embodiments, based on a total dry weight of the fat-soluble nutrient microcapsule, the fat-soluble nutrient microcapsule contains 15 to 25 mass % of the starch; for example, the fat-soluble nutrient microcapsule may contain 15 mass %, 16 mass %, 17 mass %, 18 mass %, 19 mass %, 20 mass %, 21 mass %, 22 mass %, 23 mass %, 24 mass % or 25 mass % of the starch; preferably, the fat-soluble nutrient microcapsule contains 16 to 24 mass % of the starch; and more preferably, the fat-soluble nutrient microcapsule contains 18 to 22 mass % of the starch.

### Water

The fat-soluble nutrient microcapsule according to the present disclosure may comprise a small amount of moisture, and the moisture content is not more than 5 mass % and preferably less than or equal to 3 mass % of the total mass of the fat-soluble nutrient microcapsule.

### II. Second Aspect

In the second aspect, the present disclosure provides a preparation method for the fat-soluble nutrient microcapsule according to the first aspect, comprising the following steps:
S1: mixing a fat-soluble nutrient, a hydrophilic colloid, a co-emulsifier, an optional filler, and an optional antioxidant with water, and stirring while vacuumizing to afford a crude emulsion;
S2: shearing the crude emulsion while continuously vacuumizing to afford an emulsified solution or dispersion; and
S3: subjecting the emulsified solution or dispersion to one-step granulation by a spray-starch fluidized bed drying process to afford the fat-soluble nutrient microcapsule.

By stirring and shearing while vacuumizing, the air bubbles in the emulsion are significantly reduced, which can substantially minimize the voids inside the microcapsules obtained by spray granulation and accordingly improve the density of the internal structure of the finished product of fat-soluble nutrient microcapsules. In some embodiments, the vacuum level of the entire system is greater than or equal to 0.07 MPa during vacuumization.

In some embodiments, in the step S1, the stirring is conducted at a speed of 1.0 to 1.5 m/s (e.g., 1.0 m/s, 1.1 m/s, 1.2 m/s, 1.3 m/s, 1.4 m/s or 1.5 m/s); and/or the stirring is conducted at a temperature of 60° C. to 70° C. (e.g., 60° C., 61° C., 62° C., 63° C., 64° C., 65° C., 66° C., 67° C., 68° C., 69° C. or 70° C.); and/or the stirring is conducted for 30 to 60 min (e.g., 30 min, 32 min, 34 min, 36 min, 38 min, 40 min, 42 min, 44 min, 46 min, 48 min, 50 min, 52 min, 54 min, 56 min, 58 min or 60 min).

In some embodiments, in the step S2, the shearing is conducted at a speed of greater than or equal to 19.6 m/s (e.g., 19.6 m/s or 20 m/s); and/or the shearing is conducted at a temperature of 60° C. to 65° C. (e.g., 60° C., 61° C., 62° C., 63° C., 64° C. or 65° C.); and/or the shearing is conducted for 20 to 40 min (e.g., 20 min, 22 min, 24 min, 26 min, 28 min, 30 min, 32 min, 34 min, 36 min, 38 min or 40 min).

In some embodiments, in the step S2, the emulsified solution or dispersion has a particle size Dv(90) of 1.0 to 3.0 μm.

In some embodiments, in the step S3, when the emulsified solution or dispersion is subjected to the one-step granulation by the spray-starch fluidized bed drying process, fluidized drying is conducted in stages, wherein a first stage is conducted at a temperature of 18° C. to 40° C. (e.g., 18° C., 20° C., 22° C., 24° C., 26° C., 28° C., 30° C., 32° C., 34° C., 36° C., 38° C. or 40° C.) for 10 to 20 min (e.g., 10 min, 12 min, 14 min, 16 min, 18 min or 20 min), and a second stage is conducted at a temperature of 40° C. to 60° C. (e.g., 40° C., 42° C., 44° C., 46° C., 48° C., 50° C., 52° C., 54° C., 56° C., 58° C. or 60° C.) for 40 to 60 min (e.g., 40 min, 42 min, 44 min, 46 min, 48 min, 50 min, 52 min, 54 min, 56 min, 58 min or 60 min).

In some embodiments, in the step S3, the particle size distribution of the resulting fat-soluble nutrient microcapsule ranges from 40 to 80 meshes.

### III. Third Aspect

In the third aspect, the present disclosure provides use of the fat-soluble nutrient microcapsule according to the first aspect and/or a fat-soluble nutrient microcapsule obtained by the preparation method according to the second aspect in preparation of a food or health-care product.

In some embodiments, the health-care product is a tablet.

### EXAMPLES

The embodiments of the present disclosure will be described in detail below with reference to the examples. However, a person skilled in the art will understand that the examples described below are intended only to illustrate the present disclosure and shall not be regarded as limiting the scope of the present disclosure. Where specific conditions are not indicated in the examples, conventional conditions or those recommended by manufacturers are followed. The materials or reagents used in the examples, unless otherwise specified, are all commercially-available regular products.

In the present disclosure, the pressure resistance is tested by the following method: a single microcapsule particle is placed on the tray of a texture analyzer (Yingtai Science and Technology, precision d=0.001 g); vertical pressure is applied through the probe on this capsule at 25N pressure until the capsule is 100% deformed. The morphology of the microcapsule is observed under a microscope.

In the present disclosure, the gel strength of gelatin is tested according to the determination method for gel strength of gelatin specified in the Chinese National Standard GB6783-2013.

### Example 1

1. 96 g of Vitamin A (VA) crystal, 5 g of butylated hydroxytoluene (BHT), 196 g of porcine gelatin (250 Bloom g), 87 g of fructose, 6 g of glycerin, and 10 g of sodium ascorbate were added to 500 g of pure water, and stirred while vacuumizing at 65° C. for 30 min at a stirring speed of 1.2 m/s to prepare a crude emulsion.
2. High-speed shearing was initiated while continuously vacuuming, where the vacuum level was 0.075 Mpa, the shearing speed was 19.6 m/s, and the emulsification time was 25 min, to prepare an emulsion.
3. The emulsion was subjected to one-step granulation by the spray-starch fluidized bed drying process. The fluidized drying was carried out in stages, where the first stage was conducted at a temperature of 22° C. for 15 min, and the second stage was conducted at a temperature of 56° C. for 50 min. Vitamin A microcapsules with the weight loss of less than 5% and the particle size of 40 to 80 meshes were obtained.

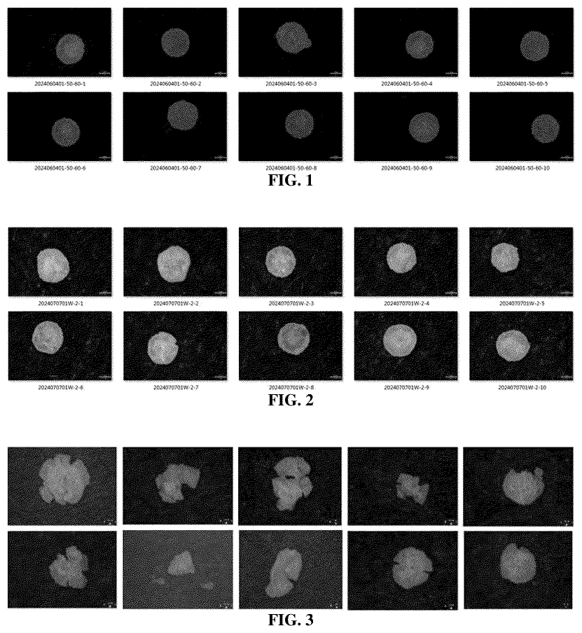

The above microcapsules were subjected to the pressure resistance test. The test results showed that the microcapsules remained intact without rupture, as shown in FIG. 1.

### Example 2

1. 96 g of Vitamin A (VA) crystal, 5 g of butylated hydroxytoluene (BHT), 196 g of porcine gelatin (250 Bloom g), 87 g of fructose, 6 g of sorbitol, and 10 g of sodium ascorbate were added to 500 g of pure water, and stirred while vacuumizing at 65° C. for 30 min at a stirring speed of 1.2 m/s to prepare a crude emulsion.
2. High-speed shearing was initiated while continuously vacuuming, where the vacuum level was 0.075 Mpa, the shearing speed was 19.6 m/s, and the emulsification time was 25 min, to prepare an emulsion.
3. The emulsion was subjected to one-step granulation by the spray-starch fluidized bed drying process. The fluidized drying was carried out in stages, where the first stage was conducted at a temperature of 22° C. for 15 min, and the second stage was conducted at a temperature of 56° C. for 50 min. Vitamin A microcapsules with the weight loss of less than 5% and the particle size of 40 to 80 meshes were obtained.

The above microcapsules were subjected to the pressure resistance test. The test results showed that part of the microcapsules was slightly ruptured, as shown in FIG. 2.

### Comparative Example 1

1. 96 g of Vitamin A (VA) crystal, 5 g of butylated hydroxytoluene (BHT), 196 g of porcine gelatin (100 Bloom g), 87 g of fructose, 6 g of glycerin, and 10 g of sodium ascorbate were added to 500 g of pure water, and stirred while vacuumizing at 65° C. for 30 min at a stirring speed of 1.2 m/s to prepare a crude emulsion.
2. High-speed shearing was initiated while continuously vacuuming, where the vacuum level was 0.075 Mpa, the shearing speed was 19.6 m/s, and the emulsification time was 25 min, to prepare an emulsion.
3. The emulsion was subjected to one-step granulation by the spray-starch fluidized bed drying process. The fluidized drying was carried out in stages, where the first stage was conducted at a temperature of 22° C. for 15 min, and the second stage was conducted at a temperature of 56° C. for 50 min. Vitamin A microcapsules with the weight loss of less than 5% and the particle size of 40 to 80 meshes were obtained.

The above microcapsules were subjected to the pressure resistance test. The test results showed that the microcapsules were severely ruptured, as shown in FIG. 3.

### Comparative Example 2

1. 96 g of Vitamin A (VA) crystal, 5 g of butylated hydroxytoluene (BHT), 196 g of Arabic gel, 87 g of fructose, 6 g of glycerin, and 10 g of sodium ascorbate were added to 400 g of pure water, and stirred while vacuumizing at 65° C. for 30 min at a stirring speed of 1.2 m/s to prepare a crude emulsion.
2. High-speed shearing was initiated while continuously vacuuming, where the vacuum level was 0.075 Mpa, the shearing speed was 19.6 m/s, and the emulsification time was 25 min, to prepare an emulsion.
3. The emulsion was subjected to one-step granulation by the spray-starch fluidized bed drying process. The fluidized drying was carried out in stages, where the first stage was conducted at a temperature of 22° C. for 15 min, and the second stage was conducted at a temperature of 56° C. for 50 min. Vitamin A microcapsules with the weight loss of less than 5% and the particle size of 40 to 80 meshes were obtained.

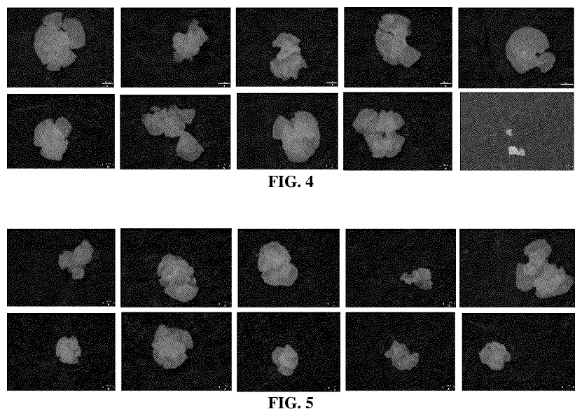

The above microcapsules were subjected to the pressure resistance test. The test results showed that the microcapsules were ruptured, as shown in FIG. 4.

### Comparative Example 3

1. 96 g of Vitamin A (VA) crystal, 5 g of butylated hydroxytoluene (BHT), 196 g of porcine gelatin (250 Bloom g), 87 g of fructose, 6 g of polyethylene glycol 400, and 10 g of sodium ascorbate were added to 500 g of pure water, and stirred while vacuumizing at 65° C. for 30 min at a stirring speed of 1.2 m/s to prepare a crude emulsion.
2. High-speed shearing was initiated while continuously vacuuming, where the vacuum level was 0.075 Mpa, the shearing speed was 19.6 m/s, and the emulsification time was 25 min, to prepare an emulsion.
3. The emulsion was subjected to one-step granulation by the spray-starch fluidized bed drying process. The fluidized drying was carried out in stages, where the first stage was conducted at a temperature of 22° C. for 15 min, and the second stage was conducted at a temperature of 56° C. for 50 min. Vitamin A microcapsules with the weight loss of less than 5% and the particle size of 40 to 80 meshes were obtained.

The above microcapsules were subjected to the pressure resistance test. The test results showed that the microcapsules were ruptured, as shown in FIG. 5.

### Test Example 1

The vitamin A microcapsules prepared in Examples 1 and 2 and Comparative Examples 1 and 2 were directly left for 12 months under the conditions of 25° C. and RH 75%. At the same time, the vitamin A microcapsules prepared in Examples 1 and 2 and Comparative Examples 1 and 2 were directly compressed into tablets and left for 12 months under the conditions of 25° C. and RH 75%. The content retention rate of vitamin A was detected. The results were shown in Table 1 below.

TABLE 1
Test Results of Content Retention Rate of Vitamin A

| | | Month | Month | Month | Month | Month |
|---|---|---|---|---|---|---|
| Items | | 1 | 3 | 6 | 9 | 12 |
| Ex. 1 | Microcapsules | 100% | 99.5% | 98.7% | 97.2% | 96.5% |
| | Microcapsule | 100% | 98.6% | 97.2% | 96.8% | 96.0% |
| | tablet | | | | | |
| Ex. 2 | Microcapsules | 100% | 98.5% | 97.7% | 96.4% | 95.3% |
| | Microcapsule | 100% | 98.2% | 96.9% | 96.2% | 95.4% |
| | tablet | | | | | |
| Comp. | Microcapsule | 98.5% | 97.6% | 96.2% | 95.4% | 94.2% |
| Ex. 1 | Microcapsule | 95.3% | 90.5% | 88.6% | 86.5% | 84.2% |
| | tablet | | | | | |
| Comp. | Microcapsules | 98.6% | 96.8% | 94.5% | 92.6% | 90.4% |
| Ex. 2 | Microcapsule | 85.0% | 76.9% | 68.6% | 60.4% | 55.0% |
| | tablet | | | | | |

The results in Table 1 showed that after the vitamin A microcapsules prepared in Examples 1 and 2 were left for 12 months under 25° C., RH75% conditions, the content retention rate of vitamin A was greater than 95%; and after they were directly compressed into tablets and left for 12 months under 25° C., RH75% conditions, the content retention rate of vitamin A was still greater than 95%, which was sufficient to meet the tableting requirements.

What is claimed is:

1. A fat-soluble nutrient microcapsule, wherein the fat-soluble nutrient microcapsule comprises the following components: a fat-soluble nutrient, a hydrophilic colloid, a co-emulsifier, and starch; wherein
the hydrophilic colloid comprises gelatin having a gel strength of 125 to 270 Bloom g; and
the co-emulsifier comprises glycerin and/or sorbitol.

2. The fat-soluble nutrient microcapsule according to claim 1, wherein the fat-soluble nutrient microcapsule comprises 30 to 60 mass % of the hydrophilic colloid and 1 to 5 mass % of the co-emulsifier, based on a total dry weight of the fat-soluble nutrient microcapsule.

3. The fat-soluble nutrient microcapsule according to claim 1, wherein the fat-soluble nutrient comprises any one or more of vitamin A, an ester of vitamin A, vitamin E, an ester of vitamin E, vitamin D2, vitamin D3, vitamin K, coenzyme Q10, curcumin, β-carotene, lutein, canthaxanthin, an ester of lutein, lycopene, astaxanthin, and a polyunsaturated fatty acid; and/or
the fat-soluble nutrient microcapsule comprises 10 to 30 mass % of the fat-soluble nutrient based on a total dry weight of the fat-soluble nutrient microcapsule.

4. The fat-soluble nutrient microcapsule according to claim 2, wherein the fat-soluble nutrient comprises any one or more of vitamin A, an ester of vitamin A, vitamin E, an ester of vitamin E, vitamin D2, vitamin D3, vitamin K, coenzyme Q10, curcumin, β-carotene, lutein, canthaxanthin, an ester of lutein, lycopene, astaxanthin, and a polyunsaturated fatty acid; and/or
the fat-soluble nutrient microcapsule comprises 10 to 30 mass % of the fat-soluble nutrient based on a total dry weight of the fat-soluble nutrient microcapsule.

5. The fat-soluble nutrient microcapsule according to claim 1, wherein the starch comprises any one or more of potato starch, corn starch, waxy corn starch, wheat starch, barley starch, rye starch, rice starch, sorghum starch, white sweet potato starch, tapioca starch, orange sweet potato starch, and legume starch; and/or
the fat-soluble nutrient microcapsule comprises 15 to 25 mass % of the starch based on a total dry weight of the fat-soluble nutrient microcapsule.

6. The fat-soluble nutrient microcapsule according to claim 4, wherein the starch comprises any one or more of potato starch, corn starch, waxy corn starch, wheat starch, barley starch, rye starch, rice starch, sorghum starch, white sweet potato starch, tapioca starch, orange sweet potato starch, and legume starch; and/or
the fat-soluble nutrient microcapsule comprises 15 to 25 mass % of the starch based on a total dry weight of the fat-soluble nutrient microcapsule.

7. The fat-soluble nutrient microcapsule according to claim 1, wherein
the fat-soluble nutrient microcapsule further comprises a filler; the filler comprises any one or more of maltodextrin, glucose, white granulated sugar, crystalline fructose, maltose, inositol, and xylitol; and/or the fat-soluble nutrient microcapsule comprises 5 to 20 mass % of the filler based on a total dry weight of the fat-soluble nutrient microcapsule; and/or
the fat-soluble nutrient microcapsule further comprises an antioxidant; the antioxidant comprises any one or more of tocopherol, butylated hydroxytoluene, butylated hydroxyanisole, tea polyphenol, propyl gallate, a rosemary extract, a licorice antioxidant, phytic acid, ascorbic acid, sodium ascorbate, D-isoascorbic acid, sodium D-isoascorbate, ascorbyl palmitate, and lecithin; and/or the fat-soluble nutrient microcapsule comprises 0.1 to 10 mass % of the antioxidant based on a total dry weight of the fat-soluble nutrient microcapsule.

8. The fat-soluble nutrient microcapsule according to claim 6, wherein
the fat-soluble nutrient microcapsule further comprises a filler; the filler comprises any one or more of maltodextrin, glucose, white granulated sugar, crystalline fructose, maltose, inositol, and xylitol; and/or the fat-soluble nutrient microcapsule comprises 5 to 20 mass % of the filler based on a total dry weight of the fat-soluble nutrient microcapsule; and/or
the fat-soluble nutrient microcapsule further comprises an antioxidant; the antioxidant comprises any one or more of tocopherol, butylated hydroxytoluene, butylated hydroxyanisole, tea polyphenol, propyl gallate, a rosemary extract, a licorice antioxidant, phytic acid, ascorbic acid, sodium ascorbate, D-isoascorbic acid, sodium D-isoascorbate, ascorbyl palmitate, and lecithin; and/or the fat-soluble nutrient microcapsule comprises 0.1 to 10 mass % of the antioxidant based on a total dry weight of the fat-soluble nutrient microcapsule.

9. The fat-soluble nutrient microcapsule according to claim 1, wherein the fat-soluble nutrient microcapsule further comprises less than or equal to 5 mass % of water.

10. The fat-soluble nutrient microcapsule according to claim 8, wherein the fat-soluble nutrient microcapsule further comprises less than or equal to 5 mass % of water.

11. A preparation method for the fat-soluble nutrient microcapsule according to claim 1,
wherein the preparation method comprises the following steps:
S1: mixing a fat-soluble nutrient, a hydrophilic colloid, a co-emulsifier, an optional filler, and an optional antioxidant with water, and stirring while vacuumizing to afford a crude emulsion;
S2: shearing the crude emulsion while continuously vacuumizing to afford an emulsified solution or dispersion; and
S3: subjecting the emulsified solution or dispersion to one-step granulation by a spray-starch fluidized bed drying process to afford the fat-soluble nutrient microcapsule.

12. A preparation method for the fat-soluble nutrient microcapsule according to claim 2, wherein the preparation method comprises the following steps:
S1: mixing a fat-soluble nutrient, a hydrophilic colloid, a co-emulsifier, an optional filler, and an optional antioxidant with water, and stirring while vacuumizing to afford a crude emulsion;
S2: shearing the crude emulsion while continuously vacuumizing to afford an emulsified solution or dispersion; and
S3: subjecting the emulsified solution or dispersion to one-step granulation by a spray-starch fluidized bed drying process to afford the fat-soluble nutrient microcapsule.

13. A preparation method for the fat-soluble nutrient microcapsule according to claim 10,
wherein the preparation method comprises the following steps:
S1: mixing a fat-soluble nutrient, a hydrophilic colloid, a co-emulsifier, an optional filler, and an optional antioxidant with water, and stirring while vacuumizing to afford a crude emulsion;
S2: shearing the crude emulsion while continuously vacuumizing to afford an emulsified solution or dispersion; and
S3: subjecting the emulsified solution or dispersion to one-step granulation by a spray-starch fluidized bed drying process to afford the fat-soluble nutrient microcapsule.

14. The preparation method according to claim 11, wherein in the step S1, the stirring is conducted at a speed of 1.0 to 1.5 m/s; and/or the stirring is conducted at a temperature of 60° C. to 70° C.; and/or the stirring is conducted for 30 to 60 min; and/or
in the step S2, the shearing is conducted at a speed of greater than or equal to 19.6 m/s; and/or the shearing is conducted at a temperature of 60° C. to 65° C.; and/or the shearing is conducted for 20 to 40 min.

15. The preparation method according to claim 13, wherein in the step S1, the stirring is conducted at a speed of 1.0 to 1.5 m/s; and/or the stirring is conducted at a temperature of 60° C. to 70° C.; and/or the stirring is conducted for 30 to 60 min; and/or
in the step S2, the shearing is conducted at a speed of greater than or equal to 19.6 m/s; and/or the shearing is conducted at a temperature of 60° C. to 65° C.; and/or the shearing is conducted for 20 to 40 min.

16. The preparation method according to claim 11, wherein in the step S3, when the emulsified solution or dispersion is subjected to the one-step granulation by the spray-starch fluidized bed drying process, fluidized drying is conducted in stages, wherein a first stage is conducted at a temperature of 18° C. to 40° C. for 10 to 20 min, and a second stage is conducted at a temperature of 40° C. to 60° C. for 40 to 60 min.

17. The preparation method according to claim 15, wherein in the step S3, when the emulsified solution or dispersion is subjected to the one-step granulation by the spray-starch fluidized bed drying process, fluidized drying is conducted in stages, wherein a first stage is conducted at a temperature of 18° C. to 40° C. for 10 to 20 min, and a second stage is conducted at a temperature of 40° C. to 60° C. for 40 to 60 min.

18. A food or a health-care product, wherein the food or the health-care product comprises the fat-soluble nutrient microcapsule according to claim 1.

19. A food or a health-care product, wherein the food or the health-care product comprises a fat-soluble nutrient microcapsule obtained by the preparation method according to claim 11.
